(19) United States
(12) Patent Application Publication
(10) Pub. No.: US 2024/0299708 A1
(43) Pub. Date: Sep. 12, 2024

(54) CATHETER ASSEMBLY WITH MICRO-TEXTURED SURFACE AND METHOD OF MANUFACTURE

(71) Applicant: Becton, Dickinson and Company, Franklin Lakes, NJ (US)

(72) Inventors: Ajay Suryavanshi, Mumbai (IN); Shishir Parasd, Ramsey, NJ (US); Himanshu Bhardwaj, Ghaziabad (IN)

(73) Assignee: Becton, Dickinson and Company, Franklin Lakes, NJ (US)

(21) Appl. No.: 18/594,723

(22) Filed: Mar. 4, 2024

Related U.S. Application Data
(60) Provisional application No. 63/450,540, filed on Mar. 7, 2023.

(51) Int. Cl. A61M 25/06; A61M 25/00; A61M 25/02

(52) U.S. Cl. CPC A61M 25/0612; A61M 25/0009; A61M 25/02; A61M 2025/0057; A61M 2025/0058; A61M 2025/006; A61M 2025/0253

(57) ABSTRACT

A catheter assembly and a method of manufacturing are described. A catheter assembly comprises a catheter body comprising a proximal end, a distal end, and a lumen formed by an inner wall of the catheter body that extends between the proximal end and the distal end along a longitudinal axis. A device may include the catheter body further comprising an external surface that extends between the proximal end and the distal end, the external surface comprising a first portion and a second portion. A device may include the second portion of the external surface including a micro-textured surface configured to spread drops of a liquid tissue adhesive across the second portion of the external surface by capillary action to form a thin film and/or micro-droplets of the liquid tissue adhesive to achieve faster curing and hence adhesion between catheter to skin.

### CROSS-REFERENCE TO RELATED APPLICATIONS

This application claims priority under 35 U.S.C. § 119(e) to U.S. Provisional Application No. 63/450,540, filed Mar. 7, 2023, the disclosures of which are incorporated herein by reference in their entirety.

### TECHNICAL FIELD

Aspects of the present disclosure relate to a catheter assembly with a micro-textured surface and methods of manufacture. Specific embodiments pertain to an intravenous catheter assembly with a micro-textured surface that facilitates securement to a patient's skin and methods of manufacture of such intravenous catheter assemblies.

### BACKGROUND

Generally, vascular access devices are used for communicating fluid with the vascular system of patients. For example, catheters are used for infusing fluid (e.g., saline solution, medicaments, and/or total parenteral nutrition) into a patient, withdrawing fluids (e.g., blood) from a patient, and/or monitoring various parameters of the patient's vascular system.

Intravenous (IV) catheter assemblies are among the various types of vascular access devices. Over-the-needle peripheral IV catheters are a common IV catheter configuration. As the name implies, an over-the-needle catheter is mounted over an introducer needle having a sharp distal tip. The introducer needle is generally a venipuncture needle coupled to a needle assembly that helps guide the needle and facilitates its cooperation with the catheter. At least the inner surface of the distal portion of the catheter tightly engages the outer surface of the needle to prevent peel back of the catheter and, thereby, to facilitate insertion of the catheter into the blood vessel. The catheter and the introducer needle are often assembled so that the sharp distal tip of the introducer needle extends beyond the distal tip of the catheter. Moreover, the catheter and needle are often assembled so that during insertion, the bevel of the needle faces up, away from the patient's skin. The catheter and introducer needle are generally inserted at a shallow angle through the patient's skin into a blood vessel.

Following catheterization, the intravenous catheter assembly is secured to the patient to prevent premature and/or unintended removal of the catheter assembly. In some instances, the clinician holds the inserted catheter assembly in place by digital pressure while preparing and applying adhesive strips to the catheter assembly. This process generally requires both hands of the clinician, and therefore the clinician commonly prepares the adhesive strips prior to inserting the catheter assembly into the patient, requiring placing the adhesive strips in a temporary location while attempting to secure the catheter assembly. This temporary location placement may provide additional opportunities for infective agents to contact the catheter assembly once the adhesive strips are in place. In other instances, a first clinician catheterizes the patient while a second clinician prepares and applies the adhesive strips to secure the inserted catheter assembly, lessening the infection risk, but greatly increasing the resources and effort required to place a catheter. Thus, the process of securing the inserted catheter assembly to the patient can be time consuming, cumbersome, and in some instances, add undue risk of infection.

Another way of securing a catheter assembly to a patient involves using a liquid tissue adhesive to adhere the hub of the catheter to the patient's skin by dispensing drops of the liquid tissue adhesive onto the catheter hub and applying the catheter hub to the patient's skin. However, the liquid tissue adhesive is dispensed in large drops that require a relatively long time to cure and for the catheter assembly to adhere to the patient's skin to secure the catheter assembly to the patient. This causes clinician to wait for long duration to ensure that the securement has been achieved. This also leads to clinician discomfort and reluctance with this mode of securement. Hence, there is an unmet need to provide catheter assemblies which will expedite the curing time with sufficient adhesion strength and to expedite the overall time spent securing the catheter assembly to a patient's skin.

### SUMMARY

On aspect of the disclosure relates to a catheter adapter assembly including: a catheter body including a proximal end, a distal end, and a lumen formed by an inner wall of the catheter body that extends between the proximal end and the distal end along a longitudinal axis; the catheter body further including an external surface that extends between the proximal end and the distal end, the external surface including a first portion and a second portion; and the second portion of the external surface including a micro-textured surface configured to spread drops of a liquid tissue adhesive across the second portion of the external surface by capillary action to form a thin film and/or form micro-droplets of the liquid tissue adhesive.

Another aspect of the disclosure relates to a method of manufacturing a catheter adapter assembly. In one embodiment, the method comprises providing a catheter body including a proximal end, a distal end, and a lumen formed by an inner wall of the catheter body that extends between the proximal end and the distal end along a longitudinal axis, the catheter body further including an external surface that extends between the proximal end and the distal end, the external surface including a first portion and a second portion; and forming a micro-textured surface on the second portion of the external surface, the micro-textured surface configured to spread drops of liquid tissue adhesive across the second portion of the external surface by capillary action to form a thin film of the liquid tissue adhesive.

### BRIEF DESCRIPTION OF THE DRAWINGS

FIG. 1A is a side view of a catheter adapter assembly according to an embodiment of the disclosure;

FIG. 1B is a bottom view of the catheter adapter assembly shown in FIG. 1A;

FIG. 1C is a bottom perspective view of the catheter adapter assembly shown in FIG. 1A;

FIG. 2 is a bottom perspective view of a catheter adapter assembly including wings;

FIG. 3 is a bottom view of the catheter adapter assembly shown in FIG. 2;

FIG. 4A is a side view of application of a liquid tissue adhesive to the catheter adapter assembly shown in FIGS. 1A-C;

FIG. 4B is a side view of application of a liquid tissue adhesive to the catheter adapter assembly shown in FIGS. 1A-C showing dispersion of the liquid tissue adhesive on the roughened surface;

FIG. 5A is a side view of application of a liquid tissue adhesive to the catheter adapter assembly with foam insert;

FIG. 5B is a side view of application of a liquid tissue adhesive to the catheter adapter assembly shown in FIG. 5A showing dispersion of the liquid tissue adhesive on the foam insert;

FIG. 6A is a side view of micro pillars;

FIG. 6B is a top view of a roughened surface comprising depressions; and

FIG. 7 is a flow chart of a method forming a catheter adapter assembly according to an embodiment of the disclosure.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

In this disclosure, a convention is followed wherein the distal end of the device is the end closest to a patient and the proximal end of the device is the end away from the patient and closest to a practitioner or clinician.

As used herein, the term “dimension” shall include the length, diameter, or width of a geometric shape or the geometrically shaped components described herein. The term “cross-sectional diameter” shall include the measurement of the longest distance or greatest distance between two points on an edge of a cross-section of an object or component with a circular or non-circular cross-section.

The two points may be located on the inside surface or outside surface of the edge of the cross-section of the object. The cross-sectional diameter of two points located on the inside surface of the edge of the cross-section of the object shall be referred to as the “inside cross-sectional diameter” and the cross-sectional diameter of two points located on the outside surface of the edge of the cross-section of an object shall be referred to as the “outside cross-sectional diameter.” It should be recognized that “cross-sectional diameter” of objects having a circular cross-section may be referred to as the “cross-sectional dimension” or “diameter” of the object. The terms “cross-sectional dimension,” “cross-sectional diameter” and “diameter” may be used interchangeably for objects having a circular cross-section.

Embodiments of the disclosure pertain to surface modification of catheter hub and/or wing. Surface modification in some embodiments involves providing a micro-textured surface on the catheter hub and/or wings. In other embodiments, surface modification of the catheter hub involves integration of foam to the catheter hub surface and/or wings. The surface modification according to one or more embodiments facilitates tissue adhesive thin film and/or micro-droplets formation via capillary action for faster curing, thus reducing the clinician wait time until the adhesive attains required bond strength.

In one or more embodiments, the catheter hub and/or wing is micro-textured or roughened on a portion of the hub surface and/or wings that contacts a patient's skin during placement of the catheter prior to intravenous insertion. Adhesive can be applied directly onto catheter hub and/or wings near the surface-modified catheter body which can be micro-patterned on the hub and/or wing body or a foam integrated into the hub and/or wing surface. Adhesive macro-droplets percolate through the roughened surface, such as a micro-pattern formed the hub and/or wings or micropores in the foam, causing the liquid tissue adhesive to quickly spread (due to capillary action) and form a thin layer and/or micro-droplets of liquid tissue adhesive at the interface of the patient's skin and hub and/or wings that contact the patient's skin. In one or more embodiments, a larger surface area provided by micro-patterned, or foam integrated catheter hub and/or wings provides quicker (curing) adhesion of the catheter assembly to a patient's skin.

Tissue adhesives for intravascular (IV) catheter application secures both the catheter to the insertion site and the hub to the skin, reducing catheter movement, migration, and dislodgement. Embodiments of the catheter assemblies described herein involves placing droplets of the liquid tissue adhesive at the insertion site and around the catheter hub and/or wings.

According to one or more embodiments, capillary action is the process of a liquid flowing in a narrow space without the assistance of, or in some embodiments, in opposition to, any external forces such as gravity. Capillary action according to some embodiments is the spontaneous upward movement of a liquid up thin tubes or fibers or micro pillars against gravity, due to adhesive and cohesive forces and surface tension, which results in the rise of the liquid in the small tubes or fibers or micro pillars According to one or more embodiments, “capillary action,” in which a liquid is moved along and/or upward, against gravity as the liquid is attracted to the internal surface of the capillaries flowing in a narrow space. For example, capillary action can be seen in the drawing up of liquids between the hairs of a paint-brush, in a thin tube, in porous materials such as a sponge. In some embodiments, capillary action occurs because of intermolecular forces between the liquid and surrounding solid surfaces. If the diameter of a tube or adjacent surfaces such as fibers or small diameter micro pillars are sufficiently small, then the combination of surface tension (which is caused by cohesion within the liquid) and adhesive forces between the liquid and the solid surfaces act to propel the liquid and form a thin film.

In one or more embodiments, tissue adhesives comprise a liquid comprising at least one liquid monomer, for example, a cyanoacrylate or a mixture of cyanoacrylates. In some embodiments, the tissue adhesive comprises 2-octyl-cyanoacrylate, 2-butyl-cyanoacrylate, or a mixture thereof. Non-limiting examples of tissue adhesives comprise Dermabond® (Ethicon US, LLC), SecurePortIV® (Adhezion Biomedical, LLC, Wyomissing, Pennsylvania), and Histoacryl® (B Braun, Sheffield, UK). In some embodiments, the tissue adhesive is a liquid monomer or a mixture of liquid monomers that undergoes an exothermic reaction upon exposure to atmospheric moisture (cyanoacrylates undergo anionic polymerization in the presence of a weak base, such as water), changing to a polymer that forms bond between the catheter and a patient's skin. In one or more embodiments, the tissue adhesive has a low viscosity (e.g., less than 200 cps at room temperature) to facilitate capillary action and forming a thin film of the tissue adhesive on the micro-textured surface to promote adhesion of the catheter assembly to the patient's skin.

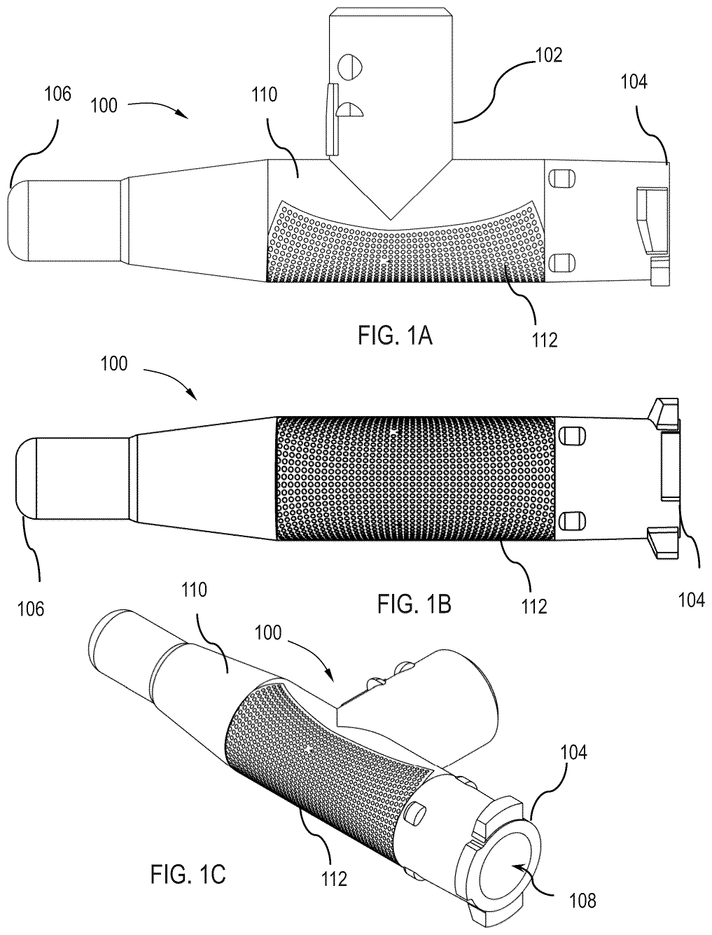

Referring now to FIGS. 1A-C, an embodiment of a catheter adapter assembly 100 is shown and comprises a catheter body 102 comprising a proximal end 104, a distal end 106, and a lumen 108 formed by an inner wall of the catheter body that extends between the proximal end 104 and the distal end 106 along a longitudinal axis of the catheter body 102. The catheter body 102 further comprising an external surface that extends between the proximal end and the distal end, the external surface comprising a first portion 110 and a second portion 112. The second portion 112 of the external surface includes a micro-textured surface configured to spread drops 120 of a liquid tissue adhesive across the second portion 112 of the external surface by capillary action to form a thin film 122 and/or micro-droplets of the liquid tissue adhesive.

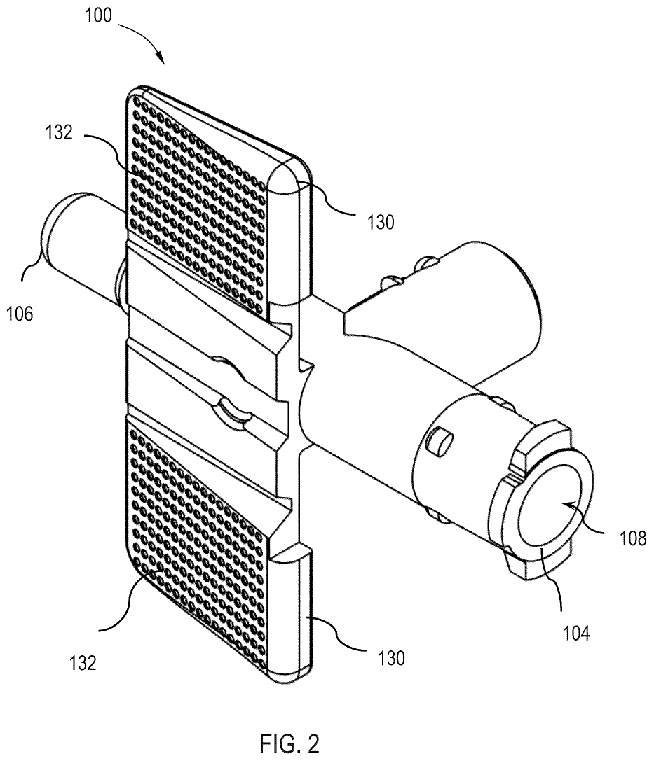

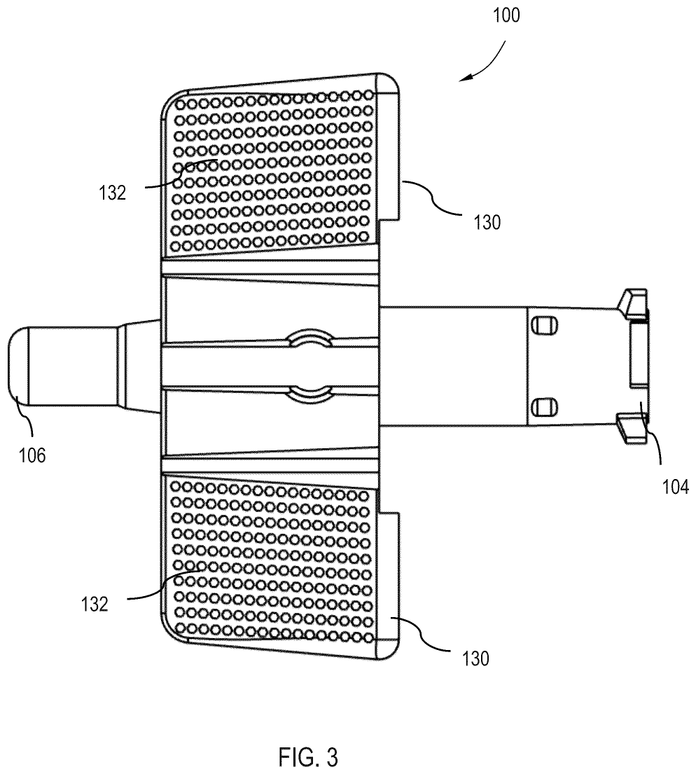

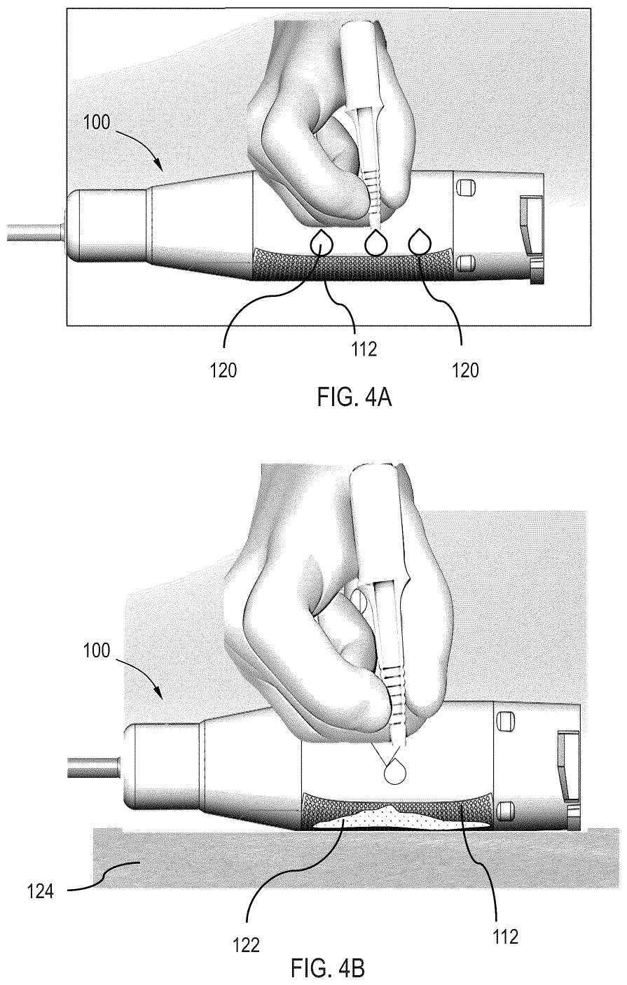

Referring to FIGS. 2 and 3 an alternative embodiment of a catheter adapter assembly 100 is shown including wings 130 having a second micro-textured surface 132 on the wings 130. The micro-textured surface is configured to spread drops of a liquid tissue adhesive across the wings 130 by capillary action to form a thin film and/or microdroplets of the liquid tissue adhesive. The thin film of the liquid tissue adhesive on second micro-textured surface 132 on the wings 130 promotes quicker curing and adhesion of the catheter assembly to the patient's skin compared to a wing surface that does not have the micro-textured surface. In some embodiments, the catheter adapter assembly 100 includes a micro-textured surface only on a second portion 112 of the catheter body 102. In some embodiments, the catheter adapter assembly 100 includes a micro-textured surface only on the wings 130 of the catheter adapter assembly 100. In other embodiments, the catheter adapter assembly 100 includes a micro-textured surface on both a second portion 112 of the catheter body 102 and on the wings 130. In other embodiment, micro-textured pattern can be present on both first (110) and second (112) surfaces of the catheter body 102. The thin film 122 of the liquid tissue adhesive formed on one or both of the micro-textured surfaces is configured to form bond with a patient's skin 124 when the catheter body is applied to the patient's skin 124.

According to one or more embodiments, “micro-textured” refers to a surface that promotes capillary action of a drops of a liquid tissue adhesive, causing the liquid tissue adhesive to spread into a thin film and/or micro-droplets so that a catheter adapter assembly having a micro-textured surface can be adhered to a patient's skin due to faster curing of the liquid tissue adhesive compared to a catheter adapter assembly having a smooth surface that is placed in contact with the patient's skin. In some embodiments, the liquid tissue adhesive comprises at least one liquid monomer having a viscosity of less than 200 cps. In some embodiments, the liquid monomer comprises at least one cyanoacrylate. In some embodiments, the micro-textured surface comprises an injection molded, micro-patterned polymer (plastic) surface. In some embodiments, the injection molded, micro-patterned polymer surface comprises a plurality of spaced pillars that promote the capillary action when in contact with the liquid tissue adhesive. In other embodiments, the micro-textured surface is formed from a micro-machined and/or laser-patterned metal mold insert via injection molding. In some embodiments, the micro-textured surface is formed from laser-patterned metal mold insert.

In one or more embodiments, a micro-pattern surface is in the form of a grid pattern, a rectangular array, a regular n-polygonal array, helical, and combinations thereof. The micro-patterned surface is designed to promote capillary action of a liquid. For example, the width d, number, and spacing s of the individual components (e.g., micro-pillars, pores, etc.) are selected and arranged to achieve a desired level of catheter adapter fixation to a patient's skin. In at least one embodiment, the micro-pattern is present at a skin-contacting surface of a catheter adapter assembly. In at least one embodiment, the individual elements of the micro-patterned surface are uniform, or the micro-patterned surface can comprise a random arrangement of micro-pillars, pores and/or other individual elements that form the micro-pattern.

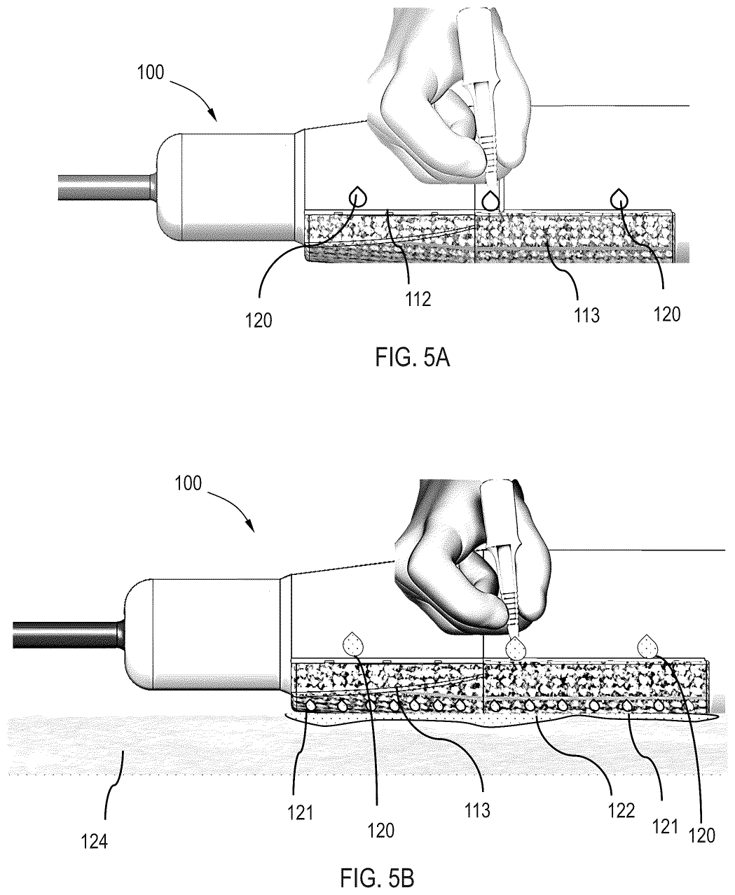

In other embodiments, the catheter adapter assembly 100 includes a micro-textured surface comprising a foam insert 113 integrated with the second portion of the external surface of the catheter body 102, as shown in FIGS. 5A and 5B. As shown in FIGS. 5A and 5B, larger drops 120 of liquid tissue adhesive dispensed by a clinician spread into smaller micro-droplets 121 and form a thin film 122 on a patient's skin 124. According to one or more embodiments, “drops” or “larger drops” refers to a volume of liquid tissue adhesive such that there 10, 15, or 20 drops per ml, and thus, each drop is in a range of from 0.05 ml to 0.1 ml, In one or more embodiments, “micro-droplet refers to a quantity of liquid tissue adhesive such that there 60 drops per ml and each microdroplet has a volume of 0.0167 ml or less. In some embodiments, a micro-is in a range of from 15 to 35% of that of macro-drop size. The thin film and/or microdroplets promote faster curing of the liquid tissue adhesive due to capillary action or wicking of the liquid tissue adhesive in pores of the foam compared with a smooth catheter body surface that is not microtextured and utilizes only drop that are larger than the micro-droplets. The foam insert 113 comprises a plurality of pores that promote capillary action when in contact with the liquid tissue adhesive. In some embodiments, the foam insert 113 is laser-welded to the second portion 112 of the external surface of the catheter body. In other embodiments, the foam insert is snap-inserted to the second portion 112 of the external surface of the catheter body.

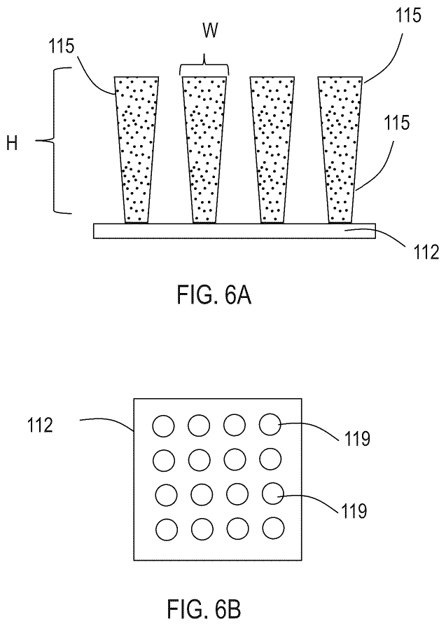

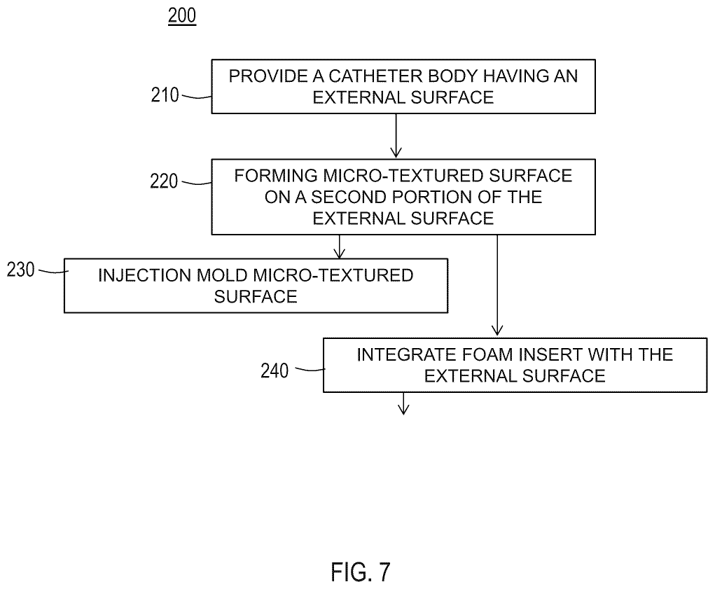

Referring to FIGS. 6A and 6B, as discussed above, a micro-textured surface 112 in some embodiments comprises micro-pillars 115 having a height H and a width W. The height of the micro-pillars 115 is greater than the width W of the micro-pillars 115 to promote capillary action between the surfaces of the pillars. In some embodiments, the pillars have an aspect ratio defined by a ratio of the height H to the width W, which is greater than 0.5, greater than 1, greater than 2, greater than 3, greater than 4, greater than 5, greater than 6, greater than 7, greater than 8, greater than 9, greater than 10 and less than 100. In some embodiments, the micro-pillars 115 have a width that is greater at the top of the micro-pillar 115 than at the bottom of the micro-pillar 115, which results in an undercut 117 near the top of the micro-pillar 114 or vice versa (width at the top smaller than at bottom of the micro-pillar 115).

Referring to FIG. 6B, the second portion 112 comprising a micro-textured surface comprises a plurality of depressions 119, which may be in the form of indentations, micro-wells, openings or other features that promote capillary action of a liquid tissue adhesive when in contact with the micro-textured surface.

In some embodiments, the height and width of the micro-pillars is 50 μm to 500 μm. Suitable heights and widths include 50 μm, 100 μm, 150 μm, 200 μm, 250 μm, 300 μm, 350 μm, 400 μm, 450 μm, and 500 μm. In other embodiments, micro-patterned elastomeric surfaces, such as those made from polymeric materials with micro-pillars of different heights and widths, for example, in a range of from 2.5 and 80 μm and heights and widths in a range of from 2.5 and 25 μm are fabricated on the surfaces of catheter adapter assemblies by injection molding using metal, ceramic or silicon molds. In one or more embodiments, spacing between individual elements such as micro-pillars are in a range of from 50 μm to 500 μm, the spacing, height and width selected to so that the micro-textured surface promotes capillary action of a liquid such as a liquid tissue adhesive when placed on the micro-textured surface. Micro-textured surfaces with aspect ratios above 0.5 promote greater adhesion than on flat surfaces without a micro-texture. Adhesion of a liquid tissue adhesive increases with decreasing micro-pillar width and increasing aspect ratio of the patterned features.

Another aspect of the disclosure pertains to a method of manufacturing a catheter adapter assembly. In one or more embodiments, a method 200 comprises at operation 210 providing a catheter body comprising a proximal end, a distal end, and a lumen formed by an inner wall of the catheter body that extends between the proximal end and the distal end along a longitudinal axis, the catheter body further comprising an external surface that extends between the proximal end and the distal end, the external surface comprising a first portion and a second portion. Such a catheter adapter is shown and described herein.

The method further comprises at operation 220 forming a micro-textured surface on the second portion of the external surface, the micro-textured surface configured to spread drops of liquid tissue adhesive across the second portion of the external surface by capillary action to form a thin film and/or micro-droplets of the liquid tissue adhesive. The method 200 according to some embodiments utilizes a catheter body comprising a polymeric material such as any material used to make catheter assemblies, for example, polypropylene. At operation 230, the method 200 further comprises injection molding the polymeric material to form the micro-textured surface comprising a micro-patterned polymer surface. In some embodiments, injection molding the micro-patterned polymer surface comprises injection molding the polymeric material on a laser-patterned metal mold insert surface. In other embodiments, the micro-patterned polymer surface formed on a laser-patterned metal mold comprises a plurality of spaced pillars that promote capillary action when in contact with a liquid tissue adhesive.

In other embodiments, the injection molding the micro-patterned polymer surface comprises injection molding the polymeric material on a micro-machined metal mold. In some embodiments, the micro-patterned polymer surface formed on the micro-machined metal mold comprises plurality of spaced pillars that promote capillary action when in contact with the liquid tissue adhesive.

In an alternate embodiment of the method, the catheter body comprises a polymeric material and the method 200 further comprises at operation 240 integrating a foam insert with the second portion of the external surface of the catheter body. In some embodiments, the foam insert comprises a plurality of pores that promote capillary action when in contact with a liquid tissue adhesive. The foam insert can be integrated by laser welding the foam insert to the second portion of the external surface of the catheter body. Alternatively, the method includes snap-fitting the foam insert to the second portion of the external surface of the catheter body.

Application of liquid tissue adhesive to a micro-textured surface of a catheter assembly as described in this disclosure causes faster spread of the adhesive on the surface of the catheter adapter assembly that is placed in contact with a patient's skin. Capillary action in the micro-textured surface results in faster curing and better adhesive strength of the catheter adapter assembly compared to a smooth surface. Advantageously, the micro-textured surface formation can be readily integrated into existing catheter adapter assembly manufacturing processes by one of several ways. In some embodiments, the molds for injection molding the catheter adapter assembly can be modified to form the desired micro-textured surface. Alternatively, a foam insert or attachment can be applied to the catheter adapter assembly after the catheter adapter assembly has been formed. The micro-textured surface, which may comprise one or more of micro-pillars, depressions, indentations, pores, and micro-channels promotes wicking and capillary action of the liquid adhesive for better liquid tissue adhesive retention and faster curing than on a smooth surface.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A catheter adapter assembly comprising:
a catheter body comprising a proximal end, a distal end, and a lumen formed by an inner wall of the catheter body that extends between the proximal end and the distal end along a longitudinal axis;
the catheter body further comprising an external surface that extends between the proximal end and the distal end, the external surface comprising a first portion and a second portion; and
the second portion of the external surface including a micro-textured surface configured to spread drops of a liquid tissue adhesive across the second portion of the external surface by capillary action to form a thin film and/or micro-droplets of the liquid tissue adhesive.

2. The catheter adapter assembly of claim 1, wherein the catheter adapter assembly comprises wings and the wings comprise a second micro-textured surface.

3. The catheter adapter assembly of claim 2, wherein the liquid tissue adhesive comprises at least one liquid monomer having a viscosity of less than 200 cps.

4. The catheter adapter assembly of claim 3, wherein the liquid monomer comprises at least one cyanoacrylate.

5. The catheter adapter assembly of claim 1, wherein the micro-textured surface comprises an injection molded, micro-patterned polymer surface.

6. The catheter adapter assembly of claim 5, wherein the injection molded, micro-patterned polymer surface comprises a plurality of spaced pillars that promote the capillary action when in contact with the liquid tissue adhesive.

7. The catheter adapter assembly of claim 5, wherein the micro-textured surface is formed from a micro-machined metal mold insert.

8. The catheter adapter assembly of claim 5, wherein the micro-textured surface is formed from laser-patterned metal mold insert.

9. The catheter adapter assembly of claim 8, wherein the injection molded, micro-patterned polymer surface comprises a plurality of spaced pillars that promote capillary action when in contact with a liquid tissue adhesive.

10. The catheter adapter assembly of claim 1, wherein the micro-textured surface comprises a foam insert integrated with the second portion of the external surface of the catheter body.

11. The catheter adapter assembly of claim 10, wherein the foam insert comprises a plurality of pores that promote capillary action when in contact with a liquid tissue adhesive.

12. The catheter adapter assembly of claim 11, wherein the foam insert is laser-welded to the second portion of the external surface of the catheter body.

13. The catheter adapter assembly of claim 11, wherein the foam insert is snap-inserted to the second portion of the external surface of the catheter body.

14. A method of manufacturing a catheter adapter assembly comprising:
providing a catheter body comprising a proximal end, a distal end, and a lumen formed by an inner wall of the catheter body that extends between the proximal end and the distal end along a longitudinal axis, the catheter body further comprising an external surface that extends between the proximal end and the distal end, the external surface comprising a first portion and a second portion; and
forming a micro-textured surface on the second portion of the external surface, the micro-textured surface configured to spread drops of liquid tissue adhesive across the second portion of the external surface by capillary action to form a thin film and/or micro-droplets of the liquid tissue adhesive.

15. The method of claim 14, wherein the catheter body comprises a polymeric material and the method further comprises injection molding the polymeric material to form the micro-textured surface comprising a micro-patterned polymer surface.

16. The method of claim 15, wherein the injection molding the micro-patterned polymer surface comprises injection molding the polymeric material on a laser-patterned metal mold insert surface.

17. The method of claim 16, wherein the micro-patterned polymer surface comprises a plurality of spaced pillars that promote capillary action when in contact with a liquid tissue adhesive.

18. The method of claim 15, wherein the injection molding the micro-patterned polymer surface comprises injection molding the polymeric material on a micro-milled metal mold.

19. The method of claim 18, wherein the micro-patterned polymer surface comprises a plurality of spaced pillars that promote capillary action when in contact with the liquid tissue adhesive.

20. The method of claim 14, wherein the catheter body comprises a polymeric material and the method further comprises integrating a foam insert with the second portion of the external surface of the catheter body.

21. The method of claim 20, wherein the foam insert comprises a plurality of pores that promote capillary action when in contact with a liquid tissue adhesive.

22. The method of claim 21, wherein the method further comprises laser welding the foam insert to the second portion of the external surface of the catheter body.

23. The method of claim 21, wherein the method further comprises snap-fitting the foam insert to the second portion of the external surface of the catheter body.
